(19) United States
(12) Patent Application Publication
(10) Pub. No.: US 2018/0028428 A1
(43) Pub. Date: Feb. 1, 2018

(54) LEAVE-IN HAIR TREATMENT COMPOSITION

(71) Applicant: Kao USA INC., Cincinnati, OH (US)

(72) Inventors: Elisabeth Cox, Cincinnati, OH (US); Christine Sarosy, Cincinnati, OH (US); Sandy Sheets, Cincinnati, OH (US)

(21) Appl. No.: 15/220,012

(22) Filed: Jul. 26, 2016

(51) Int. Cl. A61K 8/73; A61Q 5/06; A61K 8/362

(52) U.S. Cl. CPC A61K 8/732; A61K 8/362; A61Q 5/06; A61K 2800/30

(57) ABSTRACT

Leave-in hair treatment compositions, and methods of their use, deliver organic acids to the inside of hair fibers, thus providing cumulative benefits to the hair with each use, including: ease of stylability, increased volume and longer-lasting style. The leave-in hair treatment compositions utilize a combination of citric acid, succinic acid and film-forming polymers to provide the aforementioned benefits.

### TECHNICAL FIELD

The present disclosure relates to leave-in hair treatment compositions and methods of using the same to, among other things, increase hair volume, particularly fine hair volume.

### BACKGROUND

Existing leave-in hair treatments and styling products (e.g., blow-dry lotions, sprays, mousses, etc.) may contain styling polymers, i.e., film-formers, which work only on the surface of the hair. Such products coat the hair fiber with films that may make hair, particularly fine hair, feel stiff or heavy, lack movement and/or feel unnatural to the touch.

Existing leave-in hair treatment and styling products comprising film-forming polymers include:

Japanese Patent Publication Number JP 2005272391, Fukuhara et al., published Oct. 6, 2005, which describes leave-in hair cosmetics that are said to improve the luster and collectability of hair. Cellulose derivatives, such as hydroxyethyl cellulose, are identified as film-forming (set) polymers of use in the disclosed hair cosmetics.

Japanese Patent Publication Number JP 2005272399, Fukuhara et al., published Oct. 6, 2005, describes leave-in hair cosmetics that are said to have restoration/trichogenous effects on hair. Cellulose derivatives, such as hydroxyethyl cellulose, are identified as film-forming (set) polymers of use in the disclosed hair cosmetics.

Because these existing products may work only on the surface of the hair, they may not provide durability of hair style and/or volume, each of which may consequently reset with each wash. Moreover, because some of the existing products apply cellulose derivatives to the hair, a heavy or unnatural feeling may be imparted to the hair.

Some hair treatment and styling products purport to modify the hair, yet they are rinsed out of the hair shortly after application; consequently, the hair treatment and styling products, may not provide long-lasting benefits to the hair from wash to wash. For example, PCT Patent Publication Number WO 2010/049434, Dixon et al., published May 6, 2010, describes a process for straightening or relaxing hair utilizing heat and a rinse-out composition comprising weak carboxylic acids and having a pH ranging from 8 to 11.5.

Additional rinse-out hair treatment and styling products include:

PCT Patent Publication Number WO 2009/004404, Komure et al., published Jan. 8, 2007, which describes hair conditioning compositions that are particularly suited for rinse-off use. The hair conditioning compositions comprise polysaccharide polymer, aminosilicone, cationic surfactant, a high melting point fatty compound, and in some embodiments, succinic acid in unspecified amounts and/or low levels (i.e., less than 0.2%) of citric acid. The hair conditioning compositions are purported to impart a “volume-up” benefit to hair and an “ease-to-rinse” feel.

Japanese Patent Publication Number 2003286124, Kato et al., published Oct. 10, 2007, which describes a rinse-off external agent composition containing a specific phosphoric acid ester derivative. The external agent composition is purported to impart smooth and moist properties to the skin and flexibility and styling property to hair.

A need exists for a leave-in hair treatment that provides hair with long-lasting volume and ease of styling. The leave-in hair treatment compositions disclosed herein and methods of their use, advantageously convey a number of consumer benefits, which may not otherwise be achieved utilizing existing hair treatments and styling products, such as those described above. For example, the present leave-in hair treatment compositions provide hair, particularly fine hair, with cumulative benefits with each use; the cumulative benefits may last from wash-to-wash even without re-application. The cumulative benefits include, but are not limited to, ease of styling, increased hair volume and longer-lasting style. Advantageously, the benefits imparted by the present leave-in hair treatment compositions may be achieved without imparting stiffness, lack of movement and an unnatural feel, e.g., heaviness, to the hair. The aforementioned benefits may be particularly desired by consumers having fine hair.

### BRIEF SUMMARY

The present leave-in hair treatment compositions, and methods of their use, convey a number of consumer benefits through delivery of unique organic acids inside the hair in combination with delivery of film-forming starches to the surface of the hair. Without wishing to be bound by theory, it is believed that the organic acids act as structure-supporting agents inside the hair strands, thereby imparting the hair with ease of styling, increased volume and longer-lasting style during the day. Surprisingly, the organic acids may remain inside the hair strands through multiple washings, and as such, the aforementioned benefits are observed even after ceasing usage of the leave-in hair compositions. Since the delivery of organic acids into the hair strands is presently coupled with the use of starch-based film-forming polymers, the present leave-in hair treatment compositions may advantageously impart not only long-lasting effects to the hair, but may also impart the natural-feeling texture that is desirable to consumers, particularly those consumers with fine hair. Without wishing to be bound by theory, it is believed that by applying starches rather than celluloses or cellulose derivatives to the hair, a more natural, e.g., lightweight, feeling to the hair may be achieved. It is also believed that the heat sensitivity of starch, as compared to cellulose or cellulose derivatives, imparts a more natural feeling to the hair.

In some embodiments, the present disclosure is directed to leave-in hair treatment compositions having a pH of from about 2 to about 7, the hair treatment composition comprising:
(a) from about 1% to about 10% by weight of citric acid;
(b) from about 0.5% to about 10% by weight of succinic acid;
(c) from about 0.5% to about 10% by weight of polymer selected from the group: starches; modified starches; and combinations thereof; and
(d) carrier.

In some embodiments, the disclosure is directed to a hair treatment composition having a pH of greater than 2.00 and less than 3.70, the hair treatment composition comprising:
(a) a total combined weight percentage of from about 1.5% to about 10% of a mixture of citric acid and succinic acid;
(b) a total combined weight percentage of from about 1% to about 10% of polymer selected from the group: starches; modified starches; and combinations thereof; and
(c) water.

In some embodiments, the disclosure is directed to a method for increasing hair volume, the method comprising the step of applying to hair having a bending modulus, a leave-in hair treatment composition having a pH of from about 2 to about 7, the composition comprising:
(a) from about 1% to about 10% by weight of citric acid;
(b) from about 0.5% to about 10% by weight of succinic acid;
(c) from about 0.5% to about 10% by weight of polymer selected from the group: starches; modified starches; and combinations thereof; and
(d) carrier.

### BRIEF DESCRIPTION OF THE DRAWINGS

While the specification sets forth certain aspects of the present disclosure, it is believed the same will be better understood from the following description taken in conjunction with the accompanying figures in which:

FIGS. 1A-C show TOF-SIMS images of an untreated and treated hair fiber;

FIG. 2 is a diagram summarizing the texture analysis of treated vs. untreated hair fibers;

FIG. 3 is a diagram summarizing data obtained from volume and durability testing of treated vs. untreated hair fibers;

FIGS. 4A-D show photos over time of styled hair that is treated with the present leave-in hair treatment composition vs. hair treated with traditional, leave-in daily styler; and

FIGS. 5A-D show photos demonstrating the cumulative and regression effects of the present leave-in hair treatment compositions.

### DETAILED DESCRIPTION

All percentages, ratios and proportions used herein are by weight of the composition, unless otherwise specified. All average values are calculated “by weight” of the composition or components thereof, unless otherwise expressly indicated.

All references to singular characteristics or limitations of the present disclosure shall include the corresponding plural characteristic or limitation, and vice versa, unless otherwise specified or clearly implied to the contrary by the context in which the reference is made.

All combinations of method or process steps as used herein, can be performed in any order, unless otherwise specified or clearly implied to the contrary by the context in which the referenced combination is made.

As used herein, “leave-in composition” means a composition that is formulated to be applied to the hair and left in the hair for an extended period until such time as the hair is next rinsed and/or washed.

Disclosed pH values are measured at 25° C., unless otherwise expressly indicated.

The various embodiments of the leave-in hair treatment compositions according to the present disclosure may also be substantially free of any ingredient or feature described herein, provided that the remaining composition still contains all of the required ingredients or features as described herein. In this context, and unless otherwise specified, the term “substantially free” means that the selected composition contains less than a functional amount of the optional ingredient, typically less than 2.0%, including less than 1.0%, including less than 0.5%, including less than 0.1%, and also including zero percent, by weight of such optional or selected essential ingredient.

The leave-in hair treatment compositions according to the present disclosure may comprise, consist of, or consist essentially of, the elements of the compositions as described herein, as well as any additional or optional element described herein or otherwise useful in hair treatment applications.

As used herein, the term “mixtures” or “combinations” means a simple combination of materials and any compounds that may result from their combination.

As used herein, the term “untreated” means a sample to which no leave-in composition has been applied, unless otherwise specified or clearly implied to the contrary by the context in which the reference is made.

As used herein, the term “treated” means a sample to which leave-in hair composition(s) have been applied, unless otherwise specified or clearly implied to the contrary by the context in which the reference is made.

The hair treatment compositions according to the present disclosure are generally formulated as leave-in compositions. The leave-in hair treatment compositions may provide hair, particularly fine hair, with durable styling and shape creation. The leave-in hair treatment compositions may provide these and other consumer benefits while providing hair that is responsive to styling and which has a soft, natural feeling.

The present leave-in hair treatment compositions have an acidic pH, i.e., a pH of greater than about 2 and lower than about 7. In some embodiments, the leave-in hair treatment compositions have a pH in a range of from about 2 to about 7, more preferably from about 3 to about 6, more preferably from about 3 to about 5 and still more preferably from about 3 to about 4. In some embodiments, the leave-in hair treatment compositions may have a pH of greater than 2.00 and less than 3.70. In some embodiments, the leave-in hair treatment compositions may have a pH of less than 3.70.

The leave-in hair treatment compositions may comprise: organic acid; film-forming polymer; and carrier. In some embodiments, the leave-in hair treatment compositions may further comprise thickening agent. Each of the above-described components of the leave-in hair treatment compositions, as well as methods of using the same, are described in further detail below.

Organic Acid

Leave-in hair treatment compositions according to the present disclosure, comprise organic acid. The presence of organic acid in the leave-in hair treatment compositions, which have an acidic pH, may result in the penetration of hair fibers by the organic acids, which in turn, may strengthen the hair fibers. Without wishing to be bound by theory, it is believed that the organic acids strengthen a given hair fiber by forming an internal support network of multivalent salt bridges between protein chains in the hair fiber, which results in an improved ability to create lift, shapes and volume during the hair styling process. Moreover, it is believed that the present leave-in compositions are unique in that, with each use, additional organic acid may penetrate the hair fibers, which in turn, further improves styling and volume while increasing the durability of a resulting hair style.

Organic acids of use in the leave-in hair treatment compositions of the present disclosure may be selected from the group: α-carboxylic acids; multivalent carboxylic acids (e.g., 1,2-dicarboxylic acids, and/or 1,3-dicarboxylic acids); and combinations thereof. Useful α-carboxylic acids and multivalent carboxylic acids may be selected from the group of: lactic acid; citric acid; glycolic acid; tartaric acid; malonic acid; succinic acid; gluconic acid; and combinations thereof. In preferred embodiments, organic acids of use in the leave-in hair treatment compositions may comprise carboxylic acids selected from the group of: citric acid; succinic acid; and combinations thereof. In most preferred embodiments, organic acids of use in the leave-in hair treatment compositions may comprise a mixture of citric acid and succinic acid. Without wishing to be bound by theory, it is believed that in the latter embodiments of the hair treatment compositions, the combination of citric acid and succinic acid is beneficial, because citric acid provides an initial set and durability to the hair, while succinic acid provides natural feel to the hair.

In some embodiments, one or more organic acids may be present in the leave-in hair treatment compositions at from about 1.0% to about 10%, from about 1.5% to about 10%, or from about 2% to about 7%, by weight of the compositions. In some embodiments, the leave-in hair treatment compositions comprise a mixture of citric acid and succinic acid at a total combined percentage in the leave-in hair treatment compositions of from about 1.5% to about 10%, from about 2.5% to about 8%, or from about 3% to about 7%, by weight of the compositions. In some embodiments, the leave-in hair treatment compositions may comprise citric acid and succinic acid in a weight ratio of citric acid to succinic acid of: from about 20:1 to about 1:1; from about 10:1 to about 1:1; from about 5:1 to about 1:1; from about 10:1 to about 1:5; or from about 5:1 to about 1:10. In some embodiments, the leave-in hair treatment composition comprises succinic acid at from about 0.5% to about 3%, from about 1% to about 2.5%, from about 1.5% to about 2.0% by weight of the hair treatment composition. In some embodiments, the leave-in hair treatment composition comprises succinic acid at from about 0.5% to about 1.5% by weight of the hair treatment composition.

Film-Forming Polymer

Leave-in hair treatment compositions according to the present disclosure, comprise film-forming polymer. Without wishing to be bound by theory, it is believed that the presence of film-forming polymer in the leave-in hair treatment compositions delivers a tactile film to the surface of the hair fibers that may immediately impart desirable styling characteristics to the hair, without imparting stiffness, lack of movement and an unnatural feel thereto.

Film-forming polymers of use in the leave-in hair treatment compositions of the present disclosure may be selected from the group: starches (synthetic and/or plant-derived); modified starches (including silicone-modified starches and/or non-ionic modified starches); modified proteins (e.g., protein silane); and combinations thereof. In some embodiments, film-forming polymers of use in the leave-in hair treatment compositions of the present disclosure may be selected from the group: starches (synthetic and/or plant-derived); modified starches (including silicone-modified starches and/or non-ionic modified starches); and combinations thereof. In some embodiments, film-forming polymers may be selected from the group: protein silanes derived from one or more proteins selected from the group: vegetable proteins (e.g., hydrolyzed vegetable protein PG propyl silanetriol); wheat proteins; soy proteins; silk proteins; collagen; and combinations thereof; tapioca starch; rice starch (e.g., oryza (sativa) rice starch); hydroxylpropyl starch phosphate; hydroxyproyl trimonium hydrolyzed corn starch; hydrolyzed corn starch; pea starch; wheat starch; hydrolyzed vegetable protein PG propyl silanetriol; polymethylsilequioxane-modified tapioca starch; and combinations thereof.

In some embodiments, one or more film-forming polymers may be present in the leave-in hair treatment compositions at from about 0.5% to about 10%, or from about 4% to about 10%, by weight of the composition. In some embodiments, the leave-in hair treatment compositions may comprise protein-silane at from about 0.01% to about 2%, by weight of the composition. In some embodiments, the one or more film-forming polymers comprise a total combined weight percentage of from about 1% to about 10% of polymer selected from the group: starches; modified starches; and combinations thereof. In some embodiments, the leave-in hair treatment compositions may be substantially free of cellulose and/or modified cellulose, for example, hydroxyethyl cellulose.

Carrier

Leave-in hair treatment compositions according to the present disclosure, comprise a suitable carrier. Carriers are well-known in the hair treatment product formulation arts. Carriers of use in the present compositions should be safe for application to the head and hair and should be compatible with other ingredients contained in the compositions. Useful carriers may be selected from the group: water; ethanol; silicones; mineral oil; esters (e.g., isopropyl myristrate and isopropyl palmitate); other solvents commonly used in the hair care art; and mixtures thereof. In some embodiments, the carrier is water. In some embodiments, the carrier forms the balance of the leave-in hair treatment composition (i.e., the percentage of carrier present in the composition equates to 100% minus the sum total of the percentages of remaining components present in the composition).

Thickening Agent

In some embodiments, the leave-in hair treatment compositions comprise thickening agent. Thickening agents are well-known in the hair care product formulation arts. Thickening agents of use in the present compositions should be safe for application to the head and hair and should be compatible with other ingredients contained in the compositions. Useful thickening agents may be selected from the group: xanthan gum; hydroxypropyl methylcellulose; hydroxymethyl cellulose; hydroxypropyl starch phosphate; ammonium acryloyldimethytaurate/VP copolymer; PEG-150/stearyl alcohol/SMDI copolymer; long chain organic alcohols; carbomers of long chain organic alcohols; and combinations thereof. Useful long chain organic alcohols may be selected from the group of: stearyl alcohol; cetyl alcohol; behenyl alcohol; and combinations thereof.

Method of Use

The leave-in hair treatment compositions according to the present disclosure may be used via a method comprising one or more of the following steps. An effective amount of the leave-in hair treatment composition is applied to the hair, more particularly damp hair, more particularly damp hair that has been washed. In some embodiments of the method, from about 2 g to about 20 g, or from about 7 g to about 15 g, of the leave-in hair treatment composition is applied to the hair. The leave-in hair treatment composition is left in the hair, and the hair is styled as desired. In some embodiments, the method further comprises the step of blow-drying the hair.

### EXAMPLES

The following examples are illustrative only and are not intended to limit the scope of the invention in any way. Examples 1-4 are leave-in hair treatment compositions having a pH of from about 3 to about 4.

### Example 1

| Ingredient | Weight | |
|---|---|---|
| Disodium EDTA | 0.10 | g |
| Panthenol | 0.10 | g |
| Sodium Hydroxide (50%) | 1.00 | g |
| Citric Acid | 5.00 | g |
| Hydrolyzed Vegetable | 1.00 | g |
| Protein PG-Propyl | | |
| Silanetriol | | |
| Hydroxypropyltrimonium | 1.00 | g |
| Hydrolyzed Corn Starch | | |
| Polysorbate | 0.30 | g |
| Fragrance | 0.15 | g |
| Preservative | 0.50 | g |
| Water | qs 100 | g |

### Example 2

| Ingredient | Weight | |
|---|---|---|
| Sodium Hydroxide (50%) | 1.90 | g |
| Citric Acid | 5.00 | g |
| Hydrolyzed Vegetable | 1.00 | g |
| Protein PG-Propyl | | |
| Silanetriol | | |
| Hydroxypropyltrimonium | 1.00 | g |
| Hydrolyzed Corn Starch | | |
| Panthenol | 0.1 | g |
| Polysorbate | 0.30 | g |
| Fragrance | 0.15 | g |
| Preservative | 0.50 | g |
| Water | qs 100 | g |

### Example 3

| Ingredient | Weight | |
|---|---|---|
| Sodium Hydroxide (50%) | 1.90 | g |
| Citric Acid | 3.50 | g |
| Succinic Acid | 1.00 | g |
| Hydrolyzed Vegetable | 0.50 | g |
| Protein PG-Propyl | | |
| Silanetriol | | |
| Tapioca Starch | 1.50 | g |
| Hydroxypropyltrimonium | 0.50 | g |
| Hydrolyzed Corn Starch | | |
| Panthenol | 0.10 | g |
| Benzyl Alcohol | 0.25 | g |
| Triethyl Citrate | 0.15 | g |
| C12-C15 Alykl Benzoate | 2.00 | g |
| Fragrance | 0.20 | g |
| Preservative | 1.0 | g |
| Water | qs 100 | g |

### Example 4

| Ingredient | Weight | |
|---|---|---|
| Disodium EDTA | 0.10 | g |
| Sodium Hydroxide (50%) | 1.60 | g |
| Citric Acid | 3.50 | g |
| Succinic Acid | 5.00 | g |
| Hydrolyzed Vegetable | 0.50 | g |
| Protein PG-Propyl | | |
| Silanetriol | | |
| Hydroxypropyltrimonium | 0.50 | g |
| Hydrolyzed Corn Starch | | |
| Tapioca Starch | 1.50 | g |
| Polysorbate | 0.50 | g |
| Fragrance | 0.20 | g |
| Preservative | 0.20 | g |
| Water | qs 100 | g |

The leave-in hair treatment compositions, including those set forth in Examples 1-4 above, are made in a conventional manner as follows. Deionized water is added to a first vessel at room temperature. Water-soluble ingredients, including pH adjusters, salts and organic acids are added to the main vessel and mixed until homogeneous. Glycerin, thickeners and preservatives are added to a second vessel and mixed until homogenous. The mixture from the second vessel is added to the mixture in the first vessel. The resulting mixture in the first vessel is mixed and heated at 80° C. for 1 hour. At the end of 1 hour, the mixture in the first vessel is gradually cooled to 40° C., while adding any remaining ingredients and mixing the solution until homogenous resulting in a leave-in hair treatment composition. The resulting leave-in hair treatment composition, including the hair treatment compositions set forth in Examples 1-4 above, are used to treat hair, as described above.

Data

Data below demonstrate a number of the consumer benefits that may be achieved via the treatment of hair with the leave-in treatment compositions of the present disclosure.

To demonstrate that a method of treating hair with a leave-in hair treatment composition according to the present disclosure results in accumulation of organic acid in hair fibers, treated hair fibers are analyzed via time-of-flight secondary ion mass spectrometry (hereinafter, “TOF-SIMS”) as follows.

3 g of 8-inch net length, medium blonde, virgin, fine hair tresses having a range of diameters of from 45 μm-50 μm, are rinsed with water and shampooed with 0.3 g of a commercially available shampoo. The tresses are lathered for 30 seconds followed by rinsing with water for 30 seconds. Excess water is removed from the tresses by squeezing. 0.9 g of the leave-in hair treatment composition described in Example 1 above, is applied and massaged into the tresses. The tresses are placed in a 40° C. dryer box for 10 minutes. The tresses are removed from the dryer box and embedded in an ARONIX LCR D-800 resin from Toagosei Co., Ltd. (Tokyo, Japan). Cross-sections of the tresses are obtained using an ULTRACUT UTR ultramicrotome from Leica Microsystems (Buffalo Grove, Ill.). TOF-SIMS measurements are taken with a TOF-SIMS IV from Ion-tof GmbH (Munster, Germany) with a primary ion beam of 25 kV Bi₃⁺⁺. The primary ion dose density is maintained below a static limit of 10¹² ions/cm².

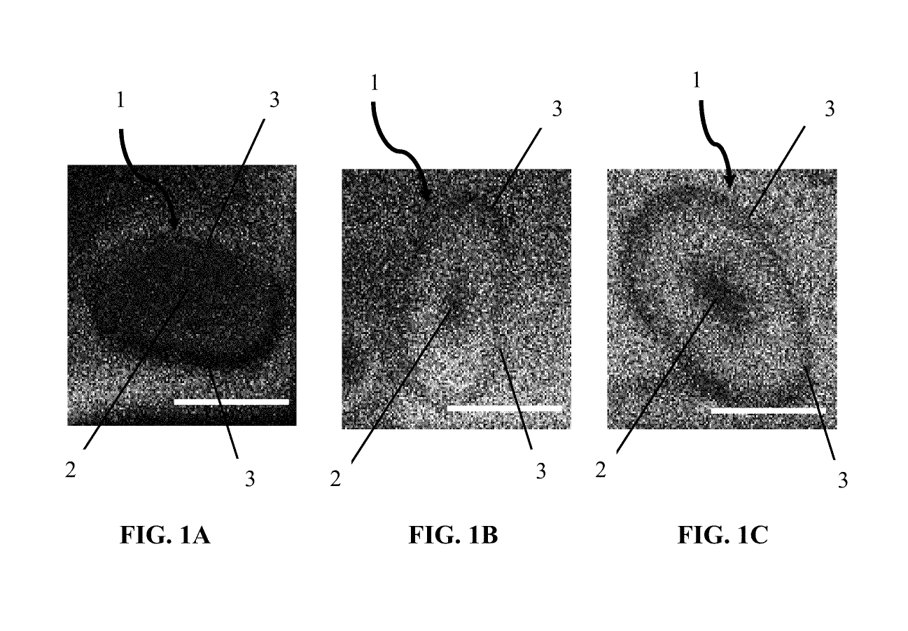

The presence of organic acid inside hair fiber from the tresses is measured at three points in time: prior to treatment with the leave-in hair treatment composition—as shown in FIG. 1A; after being treated ten times with the hair treatment composition described in Example 1—as shown in FIG. 1B; and after subsequent washing three times—as shown in FIG. 1C. Each of FIGS. 1A-1B are depicted in grayscale.

As can be seen in FIG. 1A, an untreated hair fiber (1) is predominantly dark gray throughout the cross-section of the hair fiber, including in the center (2) and along the outer edges (3) of the hair fiber. In contrast, FIG. 1B shows the hair fiber (1) after it has been treated, such that it is now predominantly light gray with only a dark gray center (2) and dark gray outer edges (3). The shift from the predominantly dark gray of the untreated fiber in FIG. 1A to the predominantly light grey of the treated fiber in FIG. 1B, is due to the presence of organic acid deposits in the hair fiber (1), which appear white. Notably, after the treated fiber is washed, the light gray of the treated fiber is retained as shown in FIG. 1C, thus demonstrating that the organic acid deposits remain accumulated inside the hair fiber (1) even after washing.

To demonstrate that stiffness of hair increases with repeated applications of a hair treatment composition according to the present disclosure, and to demonstrate that stiffness is retained after repeated washing, the texture of a sample of hair fibers is tested over a course of treatment with a leave-in hair treatment composition according to the present disclosure as follows.

5 g of 8-inch net length, medium blonde, virgin, fine hair tresses having an average diameter of 45 microns, are rinsed with water and shampooed with 0.5 g of a commercially available shampoo. The tresses are lathered for 30 seconds followed by rinsing with water for 30 seconds. Excess water is removed from the tresses by squeezing. 0.75 g of a commercially available conditioner, is applied to the tresses. The tresses are additionally rinsed for 30 seconds. Excess water is removed from the tresses by squeezing. 1.5 g of the leave-in hair treatment composition described in Example 2 is applied and massaged into the tresses. Thereafter tresses are placed in a 40° C. dryer box for 15 minutes. Immediately after removal from the drying box, a tress is mounted on a texture analyzer (e.g., the TA XT Plus from Texture Technologies (Scarsdale, N.Y.)), where it is supported on two outer points and clamped in place on one end. An arm contacts the middle portion of the tress and is lowered until there is 5 g of force on the arm. The arm is lowered an additional 20 mm, deforming the tress and measuring the maximum force on arm. The maximum force measurement is repeated 10 times on the same tress, and the average maximum force is determined by averaging the maximum force measured during the first, fifth, and tenth run of the test.

A sample of treated hair fibers and a sample of control hair fibers are measured utilizing 3-point bend testing, which quantifies changes in texture in terms of “bending modulus,” or average force required to bend the hair fibers. 3-point bend testing is measured using a texture analyzer (e.g., the TA XT Plus from Texture Technologies (Scarsdale, N.Y.).

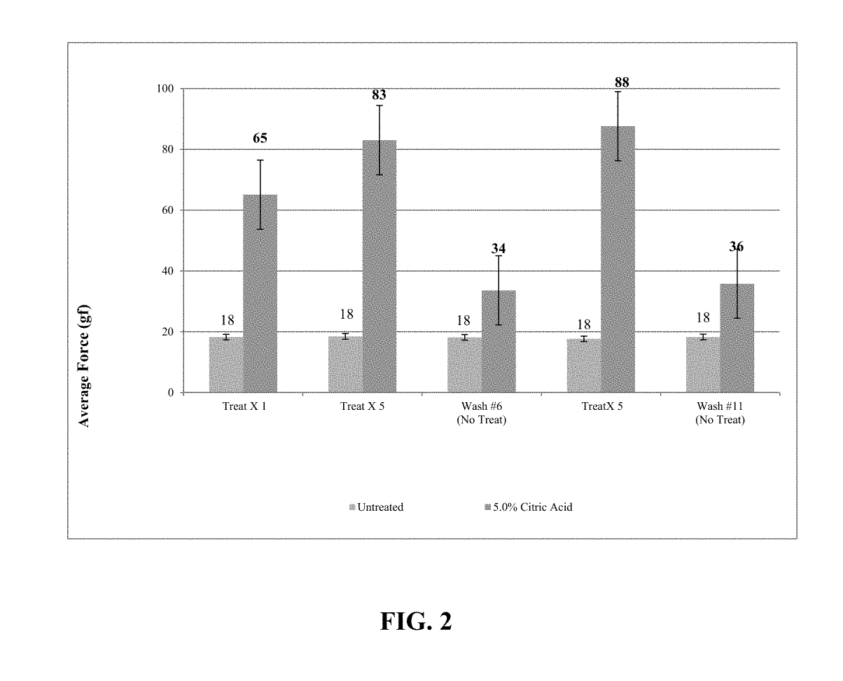

Data collected from the 3-point bend testing is summarized in FIG. 2. Moving across FIG. 2 from left to right, it is apparent that even after one treatment, the average stiffness of the treated hair fibers increases at least two-fold that of the untreated control sample.

After an additional four treatments (for a total of five treatments), the average stiffness of the treated hair fibers increases. Although a subsequent first wash of the treated sample reduces its average stiffness, it still remains significantly stiffer on average than the untreated control sample, the stiffness of which remains unchanged after the first wash.

After being treated five more times (for a total of ten treatments), the treated sample recovers the average stiffness achieved prior to the first washing. The treated sample and control sample are then subjected to a second wash. After the second wash, the average stiffness of the treated sample returns to the same level that is achieved after the first wash, yet importantly, the treated sample still remains significantly stiffer on average than the untreated control sample, the stiffness of which remains unchanged after the second wash.

To demonstrate that the treatment of hair with hair treatment compositions according to the present disclosure imparts increased volume and durability of hair styles with repeated use, as compared to untreated hair and hair treated with traditional, leave-in daily styler, root lift measurements are taken.

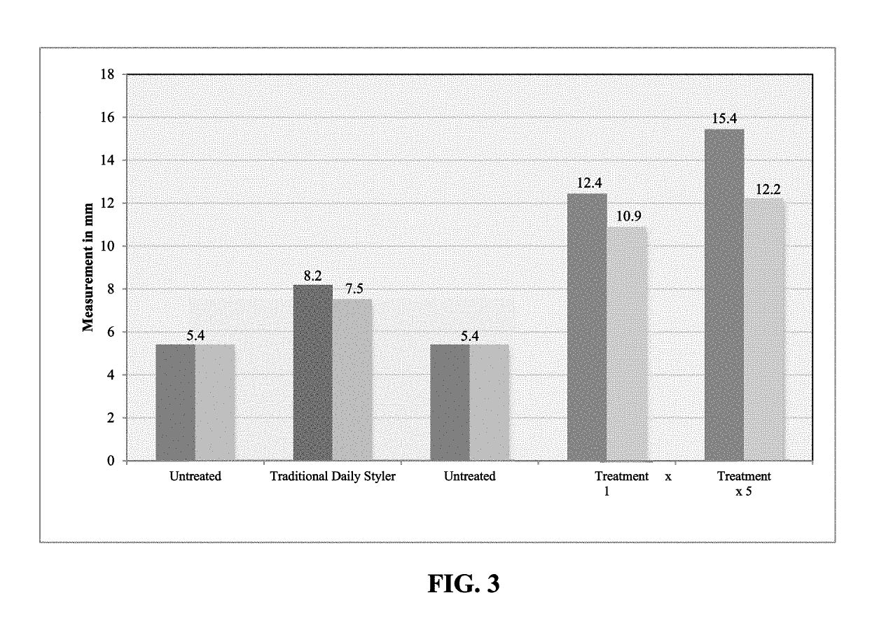

Panelist's hair is treated with shampoo, rinsed, treated with conditioner, rinsed again, and then dried with a towel. 1.5 grams of hair treatment composition described in Example 2 is applied to the hair. Hair is subsequently blow dried and styled for volume. Root lift measurements were taken using a Mitutoyo Thickness Gage from Mitutoyo America Corp. (Mason, Ohio), two inches from the front of the hair line. The above procedure is repeated five times with root lift measurements taken after the first and fifth treatment. The results of the root lift measurements are summarized in FIG. 3. Moving left to right across FIG. 3, it is clear that even after one treatment with the present leave-in hair treatment composition, the hair has substantially more volume than both untreated hair and hair treated with traditional, leave-in daily styler. Moreover, the hair volume is not only maintained, but increases with repeated applications of present leave-in hair treatment composition to the hair, which may in turn reflect increased durability of hair styles with repeated use.

To demonstrate that the leave-in hair treatment of the present disclosure provides more volume and lasting shape durability than traditional, leave-in styler, the cumulative and regression effects of each are subject to salon and mannequin testing as described below.

Salon testing of hair treated with traditional leave-in styler, and hair treated with a leave-in composition according to the present disclosure, is captured photographically immediately after treatment and re-captured 6 hours later. Panelist's hair is treated with shampoo, rinsed, treated with conditioner, rinsed again, and then dried with a towel. 3 grams of leave-in hair treatment composition described in Example 3 is applied throughout the hair and combed through. Hair is blow dried and styled.

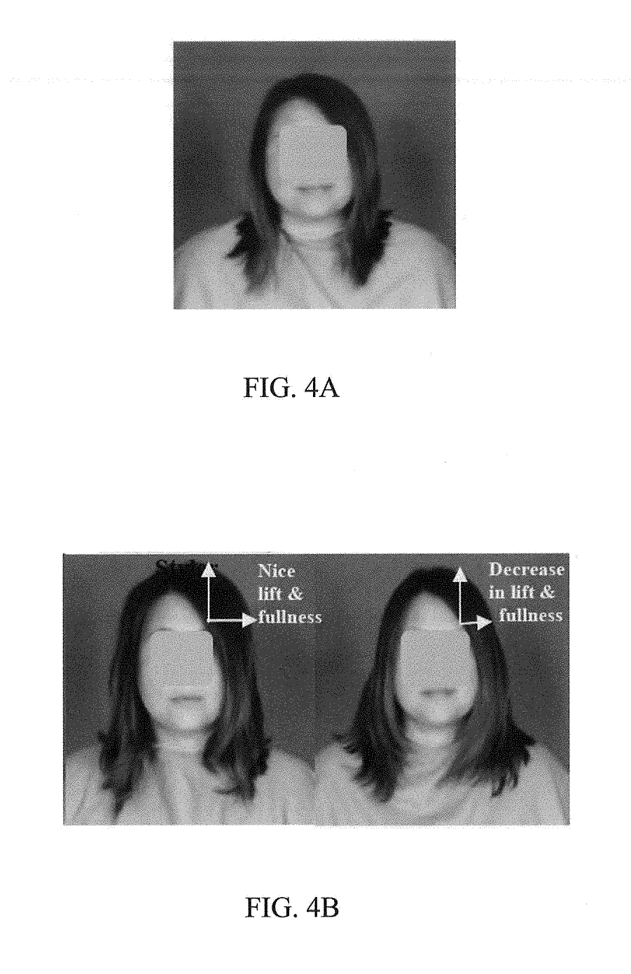

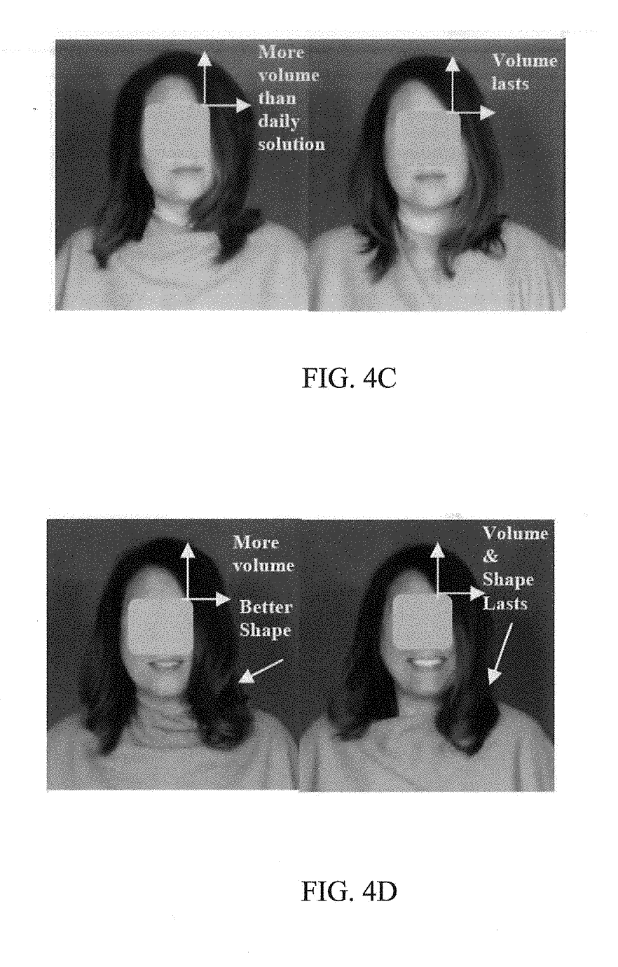

Referring now to FIG. 4A, hair is shown prior to treatment with either a traditional, leave-in daily styler or a leave-in hair treatment composition according to the present disclosure.

Moving left to right in FIG. 4B, after the hair shown in FIG. 4A is treated with traditional, leave-in daily styler, increased lift and fullness of the hair is observed (see photo on left). However, after 6 hours, the lift and fullness is visibly diminished (see photo on right).

Referring now to FIG. 4C, the hair shown in FIG. 4A is treated once with a leave-in hair treatment composition according to the present disclosure. Notably, more volume and lift is observed (see photo on left) as compared to hair that is treated with traditional, leave-in daily styler (see FIG. 4B). Moreover, more volume and lift is retained after 6 hours (see photo on right) as compared to the retained volume and lift of the hair that is treated with traditional, leave-in daily styler (see FIG. 4B).

Referring now to FIG. 4D, the hair shown in FIG. 4A is treated five times with a leave-in hair treatment composition according to the present disclosure. Once again, more volume and lift is observed (see photo on left) as compared to hair that is treated only once with the composition (see FIG. 4C). Moreover, more volume and lift is retained after 6 hours (see photo on right) as compared to the retained volume and lift of the hair that is treated with traditional, leave-in daily styler (see FIG. 4B).

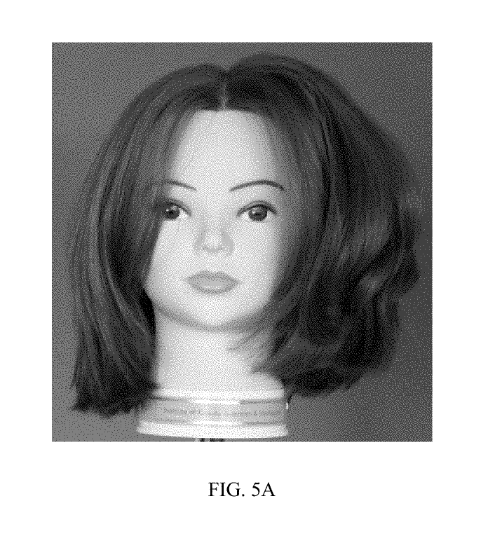

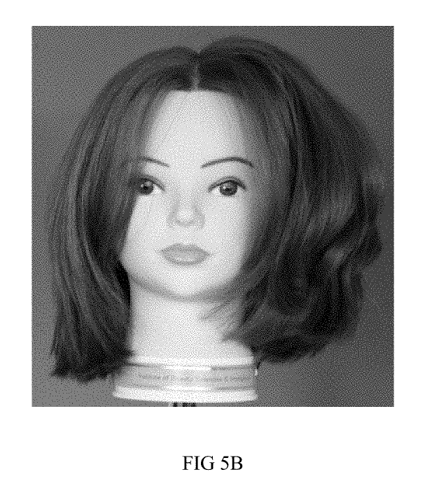

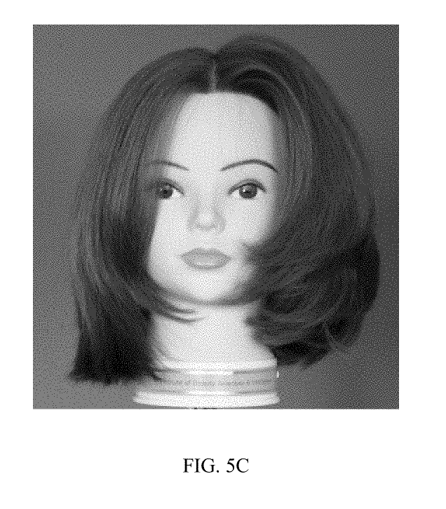

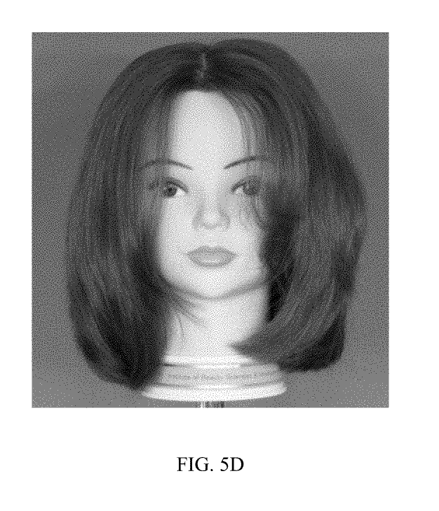

Mannequin testing of untreated, control hair, and hair that is treated with a composition according to the present disclosure, is immediately captured photographically as shown in FIGS. 5A-D. A fine-haired mannequin head having hair with an average minor hair diameter of from 40 μm-50 μm is treated with shampoo, rinsed, treated with conditioner, rinsed again, and then dried with a towel. 1.5 grams of grams of leave-in hair treatment composition described in Example 2 is applied to half of the mannequin head. The other half of the mannequin head is not treated with the leave-in hair treatment composition described in Example 2.

As can be seen in FIG. 5A, the hair that is treated only once with a leave-in hair treatment composition according to the present disclosure is observed to have increased shape and volume as compared to the untreated, control hair.

FIG. 5B shows hair after it has been treated a total of ten times with the leave-in hair treatment composition. The treated hair is observed to have increased shape and volume as compared to hair that has been treated only once (see FIG. 5A) as well as the untreated, control hair.

FIG. 5C shows both the treated and untreated, control hair of FIG. 5B after they have both been washed once. Notably, the treated hair is still observed to have increased shape and volume as compared to the untreated, control hair.

FIG. 5D shows both the treated and untreated the hair of FIG. 5C after they have both been washed a total of five times. Even after five washes, the hair is observed to retain its increased shape and volume as compared to the untreated hair.

Thus, the photos in FIGS. 5A-D visually demonstrate that with the first use, the treated hair is easier to style and shows increased shape and volume. Moreover, with increasing frequency of treatment, the hair may gain bending strength, becoming easier to shape, and the resulting styled shapes become more durable. Once treatment of the hair is stopped, the aforementioned benefits are still observed after multiple washes.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present leave-in hair treatment compositions have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A hair treatment composition having a pH of from about 2 to about 7, the hair treatment composition comprising:
(a) from about 1% to about 10% by weight of citric acid;
(b) from about 0.5% to about 10% by weight of succinic acid;
(c) from about 0.5% to about 10% by weight of polymer selected from the group: starches; modified starches; and combinations thereof; and
(d) carrier.

2. The hair treatment composition according to claim 1, wherein the composition is substantially free of modified cellulose.

3. The hair treatment composition according to claim 1, wherein the composition is a leave-in composition.

4. The hair treatment composition according to claim 1, comprising from about 2% to about 7% by weight of citric acid.

5. The hair treatment composition according to claim 1, comprising from about 2% to about 3% by weight of succinic acid.

6. The hair treatment composition according to claim 1, wherein a weight ratio of citric acid (a) to succinic acid (b) is from about 5:1 to about 1:1.

7. The hair treatment composition according to claim 1, wherein the polymer is a non-ionic modified starch.

8. The hair treatment composition according to claim 7, wherein the non-ionic modified starch is hydroxypropyl starch phosphate.

9. The hair treatment composition according to claim 1, wherein the polymer is starch selected from the group: hydroxylpropyl trimonium hydrolyzed corn starch; tapioca starch; hydrolyzed corn starch; rice starch; tapioca starch; wheat starch; pea starch; and combinations thereof.

10. The hair treatment composition according to claim 9, wherein the starch is tapioca starch.

11. The hair treatment composition according to claim 1, wherein the polymer comprises tapioca starch and a non-ionic modified starch.

12. The hair treatment composition according to claim 1, further comprising from about 0.01% to 2% of a protein-silane by weight of the hair treatment composition.

13. The hair treatment composition according to claim 12, wherein the protein-silane is derived from protein selected from the group: vegetable proteins; wheat proteins; soy proteins; silk proteins; collagen; and combinations thereof.

14. The hair treatment composition according to claim 13, wherein the protein-silane is hydrolyzed vegetable protein PG propyl silanetriol.

15. A method for increasing hair volume, the method comprising the step of applying to hair having a bending modulus, a leave-in hair treatment composition having a pH of from about 2 to about 7, the composition comprising:
(a) from about 1% to about 10% by weight of citric acid;
(b) from about 0.5% to about 10% by weight of succinic acid;
(c) from about 0.5% to about 10% by weight of polymer selected from the group: starches; modified starches; modified proteins; and combinations thereof; and
(d) carrier.

16. The method of increasing hair volume according to claim 15, further comprising the step of blow-drying the hair.

17. The method of increasing hair volume according to claim 15, further comprising increasing the bending modulus of the hair by at least about 100%.

18. The method of increasing hair volume according to claim 15, wherein the leave-in hair treatment composition is applied to damp hair.

19. The method of increasing hair volume according to claim 15, wherein the leave-in hair treatment composition has a pH of greater than 2.00 and less than 3.70.

20. A hair treatment composition having a pH of greater than 2.00 and less than 3.70, the hair treatment composition comprising:
(a) a total combined weight percentage of from about 1.5% to about 10% of a mixture of citric acid and succinic acid;
(b) a total combined weight percentage of from about 1% to about 10% of polymer selected from the group: starches; modified starches; and combinations thereof; and
(c) water.
